(19) United States
(12) Patent Application Publication
(10) Pub. No.: US 2025/0235674 A1
(43) Pub. Date: Jul. 24, 2025

(54) CATHETER INSERTION TOOL

(71) Applicant: Medtronic, Inc., Minneapolis, MN (US)

(72) Inventor: Rónán Wood, Galway (IE)

(21) Appl. No.: 18/853,683

(22) PCT Filed: Apr. 4, 2023
(86) PCT No.: PCT/US2023/017375

§ 371 Date: Oct. 2, 2024

Related U.S. Application Data
(60) Provisional application No. 63/362,499, filed on Apr. 5, 2022.

(51) Int. Cl. A61M 25/06; A61B 17/22

(52) U.S. Cl. CPC A61M 25/0662; A61B 17/22; A61B 2017/22079; A61M 2025/0681

(57) ABSTRACT

In insertion tool configured to facilitate insertion and/or introduction of a catheter into an inner lumen of a delivery sheath includes an insertion tool body defining proximal and distal openings, a side aperture, and an insertion tool lumen. The insertion tool is configured to be pre-loaded onto a catiieter such that at least a portion of a distal portion of a catheter extends out of the side aperture. For example, the insertion tool is configured, to enable the distal portion of the catheter to be preloaded within at least a portion of the insertion tool lumen in an unconstrained state. e.g., without constraining a curve and/or bend of the distal portion of the catiieter within the insertion tool lumen.

This application claims priority from and the benefit of U.S. Provisional Application No. 63/362,499, filed Apr. 5, 2022, and entitled, “CATHETER INSERTION TOOL,” the entire contents of which are incorporated herein by reference.

### TECHNICAL FIELD

This disclosure relates to medical devices.

### BACKGROUND

A medical catheter defining at least one lumen can be used with various medical procedures. For example, a medical catheter may be used to deliver a medical device or other therapy (e.g., a therapeutic agent) to a target treatment site within a patient. In some cases, a medical catheter may be used to access and treat defects in blood vessels, such as, but not limited to, lesions or occlusions in blood vessels.

### SUMMARY

This disclosure describes example insertion tools (e.g., introducer tools) configured to facilitate insertion and/or introduction of a catheter into an inner lumen of a delivery sheath, as well as medical assemblies (e.g., kits) including a catheter and one or more insertion tools. In various examples described herein, the insertion tools are configured to be pre-loaded onto a distal portion of a catheter, or in other words, with a distal portion of a catheter pre-loaded within a portion of a lumen (referred to herein as an insertion tool lumen) of the insertion tool. Example insertion tools include an aperture in a sidewall of an insertion tool housing. The aperture is open to an insertion tool lumen provides access to the lumen, e.g., in addition to proximal and distal openings to the insertion tool lumen defined by the insertion tool housing. In addition, the aperture is configured to enable at least a portion of the distal portion of a catheter to extend out of the insertion tool lumen. For example, the distal portion of the catheter may include a predetermined shape, such as a bend or a curve, which may be changed and/or altered if constrained, such as within an insertion tool lumen, for a relatively long period of time. The aperture is configured to enable the distal portion of the catheter to be preloaded within at least a portion of the insertion tool lumen in an unconstrained state, e.g., without constraining a curve and/or bend of the distal portion of the catheter within the insertion tool lumen, and also to hold the insertion tool at the distal portion of the catheter, e.g., without allowing the insertion tool to slide in a proximal direction along the catheter.

In this way, the insertion tool is configured to be delivered and/or stored in a pre-loaded configuration on a distal portion of the catheter without changing and/or altering the shape of the distal portion of the catheter. The insertion tool in the pre-loaded configuration provides protection for at least a portion of the distal portion of the catheter, and improves the accessibility of the insertion tool upon use. When it comes time to use the catheter, the distal portion of the catheter may be proximally retracted into the insertion tool lumen, such that the distal portion of the catheter does not extend through the aperture and is positioned within the insertion tool lumen. The insertion tool may then be positioned within a proximal opening of a medical device, such as an introducer sheath, and the catheter may be rotated about a longitudinal axis of the insertion tool within the insertion tool lumen such that the distal portion of the catheter bypasses the aperture in the sidewall of the insertion tool and exits the distal opening of the insertion tool and into the medical device when the catheter is subsequently advanced within the lumen of the insertion tool.

In one example, this disclosure describes a kit comprising a catheter; and an insertion tool including an insertion tool housing defining: an insertion tool lumen configured to receive at least a portion of the catheter, a proximal opening to the insertion tool lumen, and a distal opening to the insertion tool lumen. The insertion tool housing comprises a sidewall extending between the proximal and distal openings, wherein the sidewall defines an aperture, and when the at least the portion of the catheter is positioned in the insertion tool lumen, a distal portion of the catheter is configured to extend out of the insertion tool lumen via the aperture.

In another example, this disclosure describes a method of making a catheter insertion tool, the method comprising: forming an insertion tool housing comprising a proximal opening, a distal opening, and an insertion tool lumen therebetween, the insertion tool lumen being configured to receive at least a portion of a catheter, wherein a distal end of the insertion tool housing is configured to be received within an introducer lumen of an introducer sheath; and forming an aperture in a sidewall of the insertion tool, the sidewall extending between the proximal and distal openings, wherein the aperture is configured to receive a portion of the catheter to enable the catheter to extend from the insertion tool lumen to an environment external to the insertion tool lumen.

In another example, this disclosure describes a method comprising retracting a distal portion of a catheter extending through an aperture in a sidewall of an insertion tool housing of an insertion tool into an insertion tool lumen defined by the insertion tool housing, wherein the distal portion of the catheter comprises a curve; rotating the catheter to rotate the distal portion of the catheter within the insertion tool lumen such that the curve is out of alignment with the aperture; inserting a distal portion of the insertion tool housing into an introducer lumen of an introducer sheath; and advancing the catheter within the insertion tool lumen, distally past the aperture, and into the introducer lumen.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic diagram illustrating an example medical aspiration system.

FIG. 2 is a conceptual top elevation view of an example insertion tool.

FIG. 3 is a conceptual side elevation view of the example insertion tool of FIG. 2.

FIG. 4 is a conceptual bottom elevation view of the example insertion tool of FIG. 2.

FIG. 5 is a schematic cross-sectional view of the example insertion tool of FIG. 2, the cross-section being taken along line A-A in FIG. 2.

FIG. 6 is a conceptual side view of the example insertion tool of FIG. 2 with a catheter inserted in the insertion tool in a storage or pre-loaded configuration.

FIG. 7 is a conceptual side view of the example insertion tool of FIG. 2 with a catheter inserted in the insertion tool in a retracted configuration.

FIG. 8 is a conceptual side view of the example insertion tool of FIG. 2 with a catheter inserted in the insertion tool in a bypass configuration.

FIG. 9 is a conceptual side view of the example insertion tool of FIG. 2 with a catheter inserted in the insertion tool in a rotated configuration and the insertion tool inserted in an introducer sheath.

FIG. 10 is a conceptual side view of the example insertion tool of FIG. 2 with a catheter advanced through the insertion tool and the introducer sheath.

FIG. 11 is a flow diagram of an example method of using an example insertion tool.

FIG. 12 is a flow diagram of an example method of making an example insertion tool.

### DETAILED DESCRIPTION

The present disclosure describes devices, systems, and methods related to insertion of a catheter into an inner lumen of another device, such as an introducer sheath. Example devices include insertion tools configured to help protect a distal tip and/or distal portion of the catheter during storage of the catheter and during insertion of the catheter into another device, e.g., into a lumen of an introducer sheath. The insertion tools may also help guide the catheter into another medical device, such as an introducer sheath, during a medical procedure. Although examples are described primarily with respect to insertion tools configured to facilitate introduction of an aspiration catheter into an introducer sheath, a person having ordinary skill in the art reading this description will understand that the devices, systems, and methods described herein may be used for insertion of any suitable type of catheter into any suitable device, including, but not limited to, other catheters or sheaths.

Aspiration catheters are configured to deliver aspiration thrombectomy to a treatment site within a hollow anatomical structure, e.g., a blood vessel, of a patient. For example, a distal opening of an aspiration catheter may be positioned in the hollow anatomical structure near a thrombus (e.g., a blood clot or other material such as plaques or foreign bodies) and an aspiration force can be applied to a lumen of the aspiration catheter in order to draw the thrombus through the catheter lumen or via proximal withdrawal of the catheter and out of the hollow anatomical structure. The aspiration catheter is configured to navigate tortuous anatomies and may include a flexible tip and/or distal portion.

During some medical procedures, an introducer sheath is used to provide access to vasculature of a patient. For example, a lumen of the introducer sheath (“introducer lumen”) may define a pathway from a location external to the patient to a location in a blood vessel (or other hollow anatomical structure). The proximal portion of the introducer sheath can include a valve, e.g., a hemostasis valve or a seal, to help reduce the amount of blood of the patient that flows through the proximal end of the introducer sheath lumen. In some cases, during a medical procedure, a clinician uses an insertion tool to insert the catheter into the introducer sheath lumen. For example, a distal portion of the insertion tool can be inserted into the proximal end of the introducer sheath lumen and then the catheter can be inserted through a lumen of the insertion tool (“insertion tool lumen”) and into the introducer sheath lumen. The insertion tool can be relatively stiff and/or rigid (e.g., more rigid and/or having a greater stiffness than at least the distal portion of the catheter or even the entire catheter in some examples) and is configured to help protect the distal portion when the catheter is inserted into the introducer sheath lumen, e.g., as the catheter crosses the valve of the introducer sheath.

In some examples, a distal portion of an aspiration catheter may be formed with a predetermined shape, such as a bend (e.g., a curve), which may increase the aspiration force along the sidewall of a vessel and enable a user to rotate the catheter and distal portion to aspirate around the inner perimeter (e.g., circumference) of a vessel wall. The predetermined shape of a flexible distal portion of the catheter may be relatively difficult to directly advance through a valve of an introducer sheath. The insertion tools described herein can help hold the distal portion of the catheter in a more linear configuration than the predetermined non-linear shape as the distal portion of the catheter is introduced into the introducer sheath lumen.

In some instances, a catheter is housed in a medical device package with an insertion tool. For example, the insertion tool may be “pre-loaded” onto a catheter, e.g., at portion of the catheter proximal to the distal portion of the catheter. The insertion tool can be stored in the medical device package at a more proximal location than the distal portion because packaging the catheter with the insertion tool holding the distal portion of the catheter in the more linear configuration may inadvertently alter the shape of the distal portion over time. That is, a shape formed in the distal catheter portion, e.g., a distal bend of a catheter, may be changed and/or altered when constrained for a period of time, e.g., within a lumen of an insertion tool. However, because of the location of the insertion tool at the more proximal location, clinicians may be unaware of the insertion tool and, thus, may not use the insertion tool, or a medical procedure may be slowed down by the clinician having to locate and “load” the insertion tool over the distal portion of catheter.

According to examples described herein, an insertion tool is configured to protect a distal portion of a catheter during insertion within an introducer sheath and during shipping and storage of the catheter while maintaining a predetermined and/or preformed distal shape of the distal portion of the catheter. The insertion tool includes an aperture in a sidewall of the insertion configured to receive a distal portion of the catheter, e.g., into and/or out of the aperture. The aperture is open to the insertion tool lumen and is located between proximal and distal ends of the insertion tool lumen through which a catheter may be inserted into and/or out of the insertion tool lumen. That is, the aperture is located between proximal and distal openings of the insertion tool, the openings being open to the insertion tool lumen. Prior to a medical procedure, e.g., during storage in a medical device package, the catheter extends through the proximal opening and through the aperture, such that the distal portion of the catheter exits the insertion tool lumen via the aperture, e.g., rather than exiting the lumen via the distal opening of the insertion tool. The catheter may be advanced within the insertion tool such that a portion of the distal length is outside of the insertion tool via the aperture and the distal portion is unconstrained, e.g., in a relaxed state, while the catheter is within the lumen of the insertion tool.

The shape of the distal portion of the catheter, e.g., a bend, may be biased to cause the distal tip of the catheter to exit the aperture before reaching the distal opening of the insertion tool. For example, the bend may cause the distal tip to exert a force on the inner sidewall of the insertion tool when constrained within the insertion tool lumen upon being inserted in the proximal opening of the insertion tool, and when advanced to the aperture, the bend in the catheter may cause the distal tip of the catheter to exit the insertion tool lumen via the aperture, e.g., to be diverted through the aperture. The insertion tool may then provide at least some protection to the distal portion of the catheter, e.g., during delivery and storage, while maintaining the predetermined distal shape of the distal portion of the catheter.

The aperture extends around only a portion of the circumference of the insertion tool. For example, the aperture may only be on one longitudinal side of the insertion tool, e.g., a central longitudinal axis of the insertion tool dividing the insertion tool into two longitudinal sides. When it comes time insert the catheter into an introducer sheath, the distal portion of the catheter may be proximally retracted such that the distal tip of the catheter is within the insertion tool lumen. The distal portion of the catheter may then be rotated circumferentially about a longitudinal axis of the insertion tool relative to the insertion tool and/or the insertion tool may be rotated circumferentially relative to the catheter such that aperture is no longer aligned with the direction in which the distal portion of the catheter is bending. Thus, the predetermined bend of the catheter may cause the distal tip of the catheter to exert a force on the inner sidewall of the insertion tool within the insertion tool lumen at a circumferential position that is not aligned with the aperture. A portion of the insertion tool may then be inserted into a lumen of an introducer sheath. The distal portion of the catheter may then be distally advanced to exit the insertion tool lumen via the distal opening of the insertion tool rather than aperture, e.g., and into a lumen of the introducer sheath.

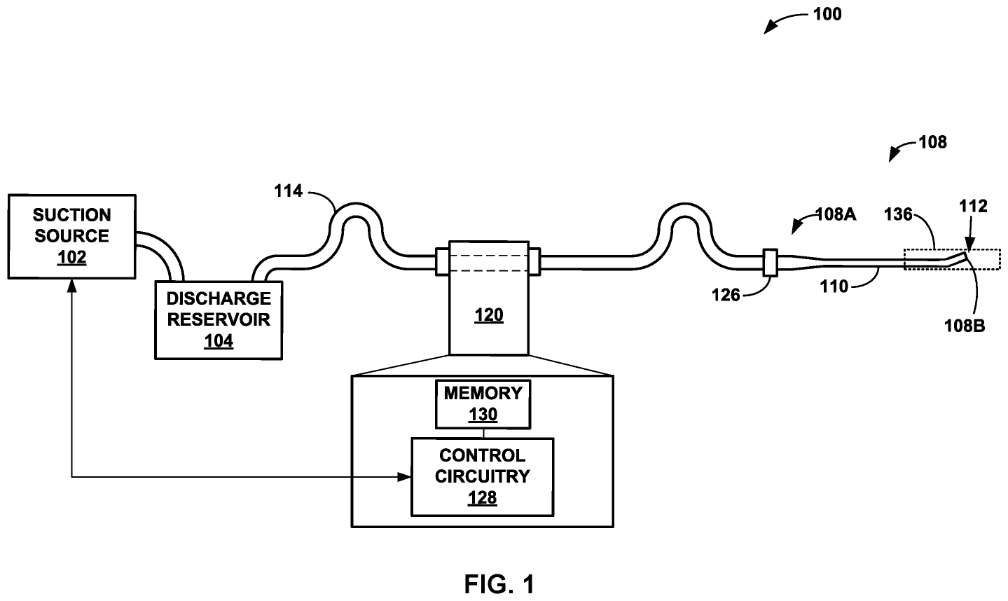

FIG. 1 is a schematic diagram illustrating an example medical system 100 including a suction source 102, a discharge reservoir 104, an aspiration catheter 108, and an insertion tool 136. Medical system 100 may be used to treat a variety of conditions, including thrombosis. Thrombosis occurs when a thrombus (e.g., a blood clot or other material such as plaques or foreign bodies) forms and obstructs vasculature of a patient. For example, medical system 100 may be used to treat deep vein thrombosis.

Medical system 100 is configured to remove fluid via catheter 108, e.g., draw fluid from catheter 108 into discharge reservoir 104, via a suction force applied by suction source 102 to catheter 108 (e.g., to a catheter lumen of catheter 108). As detailed further below, catheter 108 includes a flexible elongated body 110 defining a catheter lumen (not shown in FIG. 1) and defining a distal opening 112 to the catheter lumen. Although distal opening 112 is shown at a distal-most end 108B of catheter 108, in other examples, distal opening 112 can be more proximal to the distal end 108B. For example, distal opening 112 can be a side opening defined by a side wall of catheter 108. In some examples, catheter 108 is configured as an aspiration catheter, which can be used during an aspiration procedure to remove a thrombus or other material such as plaques or foreign bodies from vasculature of a patient. In such examples, a suction force (e.g., a vacuum) from suction source 102 is applied to proximal end 108A of catheter 108 (e.g., via hub 126) to draw a thrombus or other blockage into the catheter lumen, e.g., via distal opening 112.

To treat a patient with thrombosis, a clinician may position distal opening 112 in a blood vessel of the patient near the thrombus or other occlusion, and apply a suction force (also referred to herein as suction, vacuum force, or negative pressure) to catheter 108 (e.g., to one or more lumens of the catheter) to engage the thrombus with suction force at distal opening 112. For example, suction source 102 can be configured to create a negative pressure within the catheter lumen of catheter 108 to draw a fluid, such as blood, an aspiration fluid, more solid material, or a combination thereof, into the catheter lumen via distal opening 112 of catheter 108. The negative pressure within the catheter lumen can create a pressure differential between the catheter lumen and the environment external to at least a distal portion of catheter 108 that causes fluid and other material to be introduced into the catheter lumen via distal opening 112. For example, the fluid may flow from patient vasculature, into the catheter lumen via distal opening 112, and subsequently through aspiration tubing 114 (also referred to herein as “vacuum tube 114”) into discharge reservoir 104.

Once distal opening 112 of aspiration catheter 108 has engaged the thrombus, the clinician may remove aspiration catheter 108 with the thrombus held within distal opening 112 (or appended to a distal-most end of elongated body 110 that defines distal opening 112), or suction off pieces of the thrombus (or the thrombus as a whole) until the thrombus is removed from the blood vessel of the patient, either through the catheter lumen of aspiration catheter 108 itself, and/or through the lumen of an outer catheter (or “sheath”) in which aspiration catheter 108 is at least partially positioned. The outer catheter can be, for example, a guide catheter configured to provide additional structural support to aspiration catheter 108.

As used herein, “suction force” is intended to include, within its scope, related concepts such as suction pressure, vacuum force, vacuum pressure, negative pressure, fluid flow rate, and the like. A suction force can be generated by a vacuum, e.g., by creating a partial vacuum within a sealed volume fluidically connected to a catheter, or by direct displacement of liquid in a catheter or tubing via (e.g.) a peristaltic pump, or otherwise. Accordingly, suction forces or suction as specified herein can be measured, estimated, computed, etc. without need for direct sensing or measurement of force. A “higher,” “greater,” or “larger” (or “lower,” “lesser,” or “smaller”) suction force described herein may refer to the absolute value of the negative pressure generated by the suction source on catheter 108 or another component, such as discharge reservoir 104.

In some examples, suction source 102 can comprise a pump and/or an aspiration pump (also referred to herein as “pump 102” or “vacuum source 102”). The suction source 102 can include one or more of a positive displacement pump (e.g., a peristaltic pump, a rotary pump, a reciprocating pump, or a linear pump), a direct-displacement pump (e.g., a peristaltic pump, or a lobe, vane, gear, or piston pump, or other suitable pumps of this type), a direct-acting pump (which acts directly on a liquid to be displaced or a tube containing the liquid), an indirect-acting pump (which acts indirectly on the liquid to be displaced), a centrifugal pump, and the like. An indirect-acting pump can comprise a vacuum pump, which displaces a compressible fluid (e.g., a gas such as air) from the evacuation volume (e.g., discharge reservoir 104, which can comprise a canister), generating suction force on the liquid. Accordingly, the evacuation volume (when present) can be considered part of the suction source. In some examples, suction source 102 includes a motor-driven pump, while in other examples, suction source 102 can include a syringe configured to be controlled by control circuitry 128, and mechanical elements such as linear actuators, stepper motors, and the like. As further examples, the suction source 102 could comprise a water aspiration venturi or ejector jet.

Control of suction source 102 can comprise control, operation, and the like, of any one or combination of the component(s) making up the suction source. Accordingly, in examples in which suction source 102 includes a pump and an evacuation volume, control of the suction source can comprise control of only the pump, of only the evacuation volume, or of both of those components. As in examples in which suction source 102 includes only a pump, control of suction source 102 comprises control of the pump.

In some examples, aspiration system 100 includes control circuitry 128 configured to control a suction force applied by suction source 102 to the catheter lumen. For example, control circuitry 128 can be configured to directly control an operation of suction source 102 to vary the suction force applied by suction source 102 to the lumen of catheter 108, e.g., by controlling the motor speed, or stroke length, volume or frequency, or other operating parameters, of suction source 102. For instance, control circuitry 128 may vary the suction force by intermittently varying the aspiration force, by periodically varying the aspiration force, or by pulsing the aspiration force, as a few non-limiting examples.

Control circuitry 128, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, control circuitry 128 may include multiple components, such as any combination of one or more microprocessors, one or more DSPs, one or more ASICs, or one or more FPGAs, as well as other discrete or integrated logic circuitry, and/or analog circuitry. In some examples, control circuitry 128 is or includes a “smart” device or system, including, but not limited to, a robotic device (e.g., a robotic surgical system), a device configured to operate with the aid of artificial intelligence (AI), a virtual reality (VR) system configured to aid a clinician with the medical procedure, cloud-based interfaces for data processing and/or data storage, or any combination thereof. In some examples, control circuitry 128 may further include, additionally or alternatively to electric-based processors, one or more controls that operate using fluid motion power (e.g., hydraulic power) in combination with or in addition to electricity. For example, control circuitry 128 can include a fluid circuit comprising a plurality of fluid passages and switches arranged and configured such that, when a fluid (e.g., a liquid or gas) flows through the passages and interacts with the switches, the fluid circuit performs the functionality of control circuitry 128 described herein.

Memory 130 may store program instructions, such as software, which may include one or more program modules, which are executable by control circuitry 128. When executed by control circuitry 128, such program instructions may cause control circuitry 128 to provide the functionality ascribed to control circuitry 128 herein. The program instructions may be embodied in software and/or firmware. Memory 130, as well as other memories described herein, may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media.

In some examples, but not all examples, a distal portion of elongated body 110 of catheter 108 includes an expandable distal portion (not shown) configured to expand radially outward to widen distal opening 112 for engaging with a thrombus.

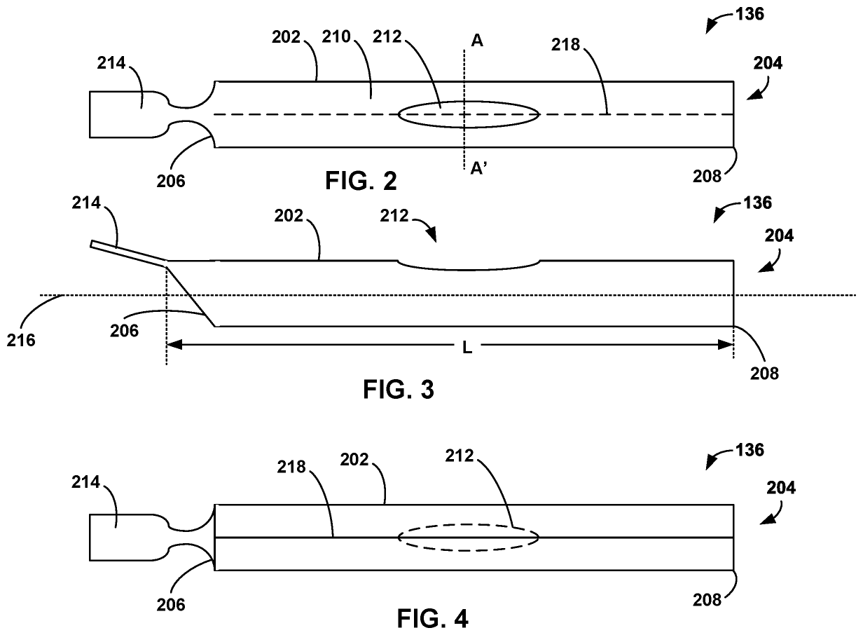

As detailed further below with respect to FIGS. 2-11, medical system 100 includes insertion tool 136 configured to aid in insertion of catheter 108 into an inner lumen of another device, such as an introducer sheath, and ultimately into the vasculature of the patient. At least a portion of insertion tool 136 may be inserted into the proximal end of an introducer sheath lumen, and catheter 108 may then be inserted through a lumen of insertion tool 136 and into the introducer sheath lumen. Insertion tool 136 may be relatively rigid and configured to help protect a distal portion of catheter 108 when catheter 108 is inserted into the introducer sheath. Insertion tool 136 is also configured to protect a distal portion of catheter 108 during shipping and storage of catheter 108 while maintaining a predetermined and/or preformed distal shape of the distal portion of catheter 108.

In some examples, catheter 108 and insertion tool 136 are packaged, delivered, and stored separately from at least some of the other components of medical system 100, e.g., as a kit. In some examples, catheter 108 and insertion tool 136 may be single-use components of medical system 100 and may be replaced with a new catheter 108 and insertion tool 136 for each procedure, e.g., unpackaged from sterile packaging and coupled to suction source 102 (directly or indirectly) via aspiration tubing 114 at the start of a procedure. In some examples, catheter 108 is inserted into insertion tool 136 in the pre-loaded configuration and then packaged within a package housing (e.g., packaging such as a sterile bag or other container). In some examples, catheter 108 and insertion tool 136 may be sterilized, before or after being positioned in the pre-loaded configuration, within a sterilized package housing.

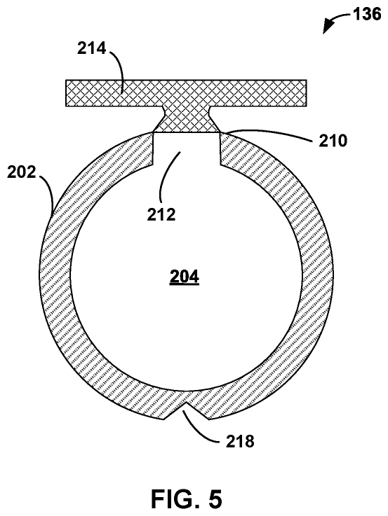

FIGS. 2-5 are different conceptual views of an example insertion tool 136. FIG. 2 is a conceptual top elevation view of example insertion tool 136, FIG. 3 is a conceptual side elevation view of example insertion tool 136, FIG. 4 is a conceptual bottom elevation view of example insertion tool 136, and FIG. 5 is a conceptual cross-sectional view of insertion tool 136, the cross-section being taken along line A-A in FIG. 2.

As shown in FIGS. 2-5, insertion tool 136 includes a housing 202 defining an insertion tool lumen 204 extending between proximal opening 206 and distal opening 208. Insertion tool lumen 204 is configured to receive at least a distal portion of a catheter, e.g., catheter 108. Housing 202 includes sidewall 210 extending between proximal opening 206 and distal opening 208 and defining aperture 212. Proximal opening 206 and distal opening 208 are shown to be at the proximal-most and distal-most ends, respectively, of housing 202 in the example shown in FIGS. 2-5, but can be at other locations along housing 202 in other examples. In the example shown, housing 202 is tubular, however, in other examples housing 202 may be any suitable shape. Aperture 212 is open to insertion tool lumen 204, and is configured to receive at least a portion of a distal portion of catheter 108 into and/or out of insertion tool lumen 204.

Insertion tool 136 is configured to receive at least a portion of catheter 108 via any of proximal opening 206, distal opening 208, and aperture 212. For example, a distal portion of catheter 108 may be inserted into proximal opening 206 and distally advanced through insertion tool lumen 204 to extend out of distal opening 208, or the distal portion of catheter 108 may be inserted into proximal opening 206 and distally advanced through insertion tool lumen 204 to extend out of aperture 212. In some examples, insertion tool 136 is configured such that, when a distal portion of catheter 108 is positioned within insertion tool lumen 204, a portion of the distal portion of catheter 108 extends out of insertion tool lumen 204 via aperture 212, e.g., such a catheter 108 having a bent (e.g., curved) configuration is received within insertion tool 136 in an unconstrained configuration. For example, a bend of the distal portion of catheter 108 may be proximate to aperture 212 such that a portion of catheter 108 proximal to the bend is within insertion tool lumen 204 and a portion of catheter 108 distal to the bend and/or curve extends out of insertion tool lumen 204 via aperture 204, e.g., in a “pre-loaded” configuration as illustrated in FIG. 7 and described in further detail below.

In some examples, once catheter 108 and insertion tool 136 are in a pre-loaded configuration, catheter 108 and insertion tool 136 do not move very much relatively to each other and a threshold amount of force (e.g., purposeful application of a proximal pulling force on catheter 108) is required to retract the distal portion of catheter 108 into insertion tool lumen 204, e.g., in the proximal direction. For example, catheter 108 may retain and/or hold insertion tool 136 with a threshold amount of force to prevent insertion tool 136 from slipping off of catheter 108 and/or moving proximally along catheter 108 (e.g., proximally “sliding down” catheter 108). Similarly, insertion tool 136 may retain and/or hold a distal portion of catheter 108 with a threshold amount of force to prevent catheter 108 from proximally retracting from insertion tool lumen 204 (e.g., “slipping out from” insertion tool 136) or further extending through aperture 212.

Aperture 212 has any suitable shape configured to receive a portion of catheter 108 there through. In some examples, aperture 212 has an elliptical cross-sectional shape. For example, a length of aperture 212 along longitudinal axis 216 of housing 202 may be greater than a width of aperture 212 in a width direction transverse to longitudinal axis 216 (e.g., a circumferential direction extending along an outer surface of housing 202). In some examples, catheter 108 has a circular cross-sectional shape, and the bend and/or curve of the distal portion of catheter 108 may cause catheter 108 to extend out of insertion tool lumen 204 through aperture 212 at an angle with respect to longitudinal axis 216. Aperture 212 may be sized in the width direction to be substantially the same or slightly wider than the outside diameter of catheter 108, and the length of aperture 212 may then be longer to accommodate for catheter 108 extending through aperture 212 at an angle. Thus, in some cases, the length of aperture 212 can be selected based on the amount of curvature of catheter 108.

Housing 202 can be formed from any suitable material. In some examples, housing 202 comprises a polymer material, e.g., nylon, polycarbonate, polyester, polypropylene, a vinyl, or a thermoset plastic or the like, or a vinyl alcohol polymer or polyvinyl alcohol, or a metal, or any combination thereof. A material of housing 202 can be selected, in some examples, such that housing 202 is relatively rigid, e.g., a stiffness of housing 202 may be greater than a stiffness of at least the distal portion of catheter 108 intended to be housed within insertion tool lumen 204. In some examples, housing 202 is configured to be substantially incompressible when received within an introducer lumen of the introducer sheath and/or introducer valve. For example, housing 202 may have rigidity and/or stiffness configured to be interference fit within the lumen of the introducer sheath substantially without buckling and/or compressing radially inwards. In some examples, housing 202 may be configured to open a valve (e.g., a seal) within the lumen of the introducer sheath or introducer valve when housing 202 is inserted through the valve. For example, housing 202 may have rigidity and/or stiffness configured to cause a seal of the introducer sheath to open and/or widen as insertion tool 136 is inserted into the introducer sheath and/or valve.

In some examples, housing 202 may be substantially transparent or substantially translucent. For example, housing 202 may be configured such that catheter 108 may be visibly seen through housing 202 when at least a portion of catheter 108 is within insertion tool lumen 204.

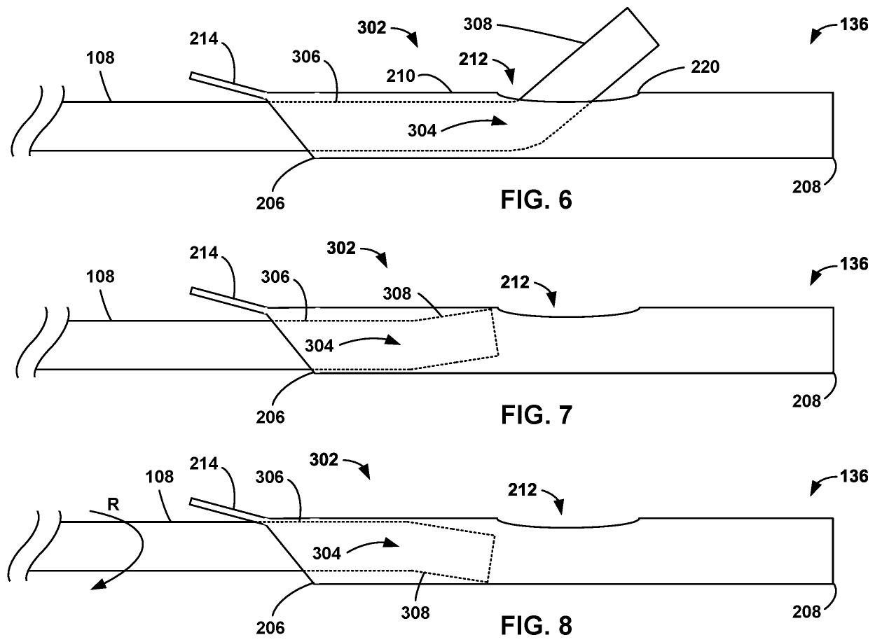

Housing 202 can have any suitable dimensions. For example, housing 202 may be long enough to enable a curve of catheter 108 (e.g., curve 304 of FIG. 6) to extend through aperture 212 and short enough to enable housing 202 to be stored over catheter 108, e.g., in packaging for delivery and/or storage. In some examples, the cross-sectional shape of housing 202 (FIG. 5) may be circular, elliptical, or any suitable shape. In some examples, the cross-sectional shape of housing 202 may define a cross-sectional shape of insertion tool lumen 204 (e.g., an inner diameter of housing 202) that is the same as the cross-sectional shape of the outer diameter of housing 202, e.g., both circular with different diameters. In other examples, housing 202 may define inner and outer cross-sectional shapes that are different, e.g., a circular inner diameter (insertion tool lumen 204) and an elliptical, square, rectangular, triangular, or the like, outer perimeter cross-sectional shape. In some examples, housing 202 may define an inner diameter of insertion tool lumen 204 that is substantially equal to, or slightly larger than, an outer diameter of catheter 108, e.g., at least a distal portion of catheter 108. In some examples, housing 202 may define an outer diameter (e.g., including sidewall 210) that may be substantially equal to or less than an inner diameter of the lumen of the introducer sheath or introducer valve.

In some examples, a longitudinal length L of housing 202, measured in a direction parallel to longitudinal axis 216, is greater than or equal to 40 millimeters (mm). In the example shown, housing 202 has a length L, which does not include tab 214 (described in further detail below). In some examples, length L includes tab 214. In some examples, a distal portion of housing 202 is tapered, e.g., to ease inserting insertion tool 136 into the introducer sheath and/or valve. For example, an outer diameter of a distal portion of housing 202 may be tapered such that an outer diameter of the distal end of the distal portion (e.g., proximate to distal opening 208) is less than an outer diameter housing 202 at a longitudinal position proximal to the distal end (e.g., closer to proximal opening 206). In some examples, the outer diameter of housing 202 may be distally tapered via reducing the thickness of housing 202 and/or sidewall 210, e.g., and without changing the diameter of insertion tool lumen 204.

In the example shown, housing 202 includes tab 214 extending from housing 202 and configured to be gripped a user, e.g., to control motion of housing 202 when being gripped. For example, tab 214 may extend away from longitudinal axis 216 of housing 202 at the proximal end of the length of housing 202. In some examples, tab 214 is integrally formed with housing 202, and in other examples tab 214 is formed separate from and attached to housing 202, e.g., positioned and attached to a proximal end of housing 202. Tab 214 may comprise a length, width, area, thickness, and/or rigidity configured to allow a user to manipulate insertion tool 136 via gripping and/or exerting a force on tab 214. In some examples, tab 214 is configured to allow the user to hold insertion tool 136 steady while advancing and/or retracting catheter 108 through insertion tool lumen 204. In some examples, tab 214 is configured to allow the user to otherwise move insertion tool 136 relative to catheter 108 and/or an introducer sheath. For example, after using insertion tool 136 to insert at least a portion of catheter 108 into the introducer sheath and/or vasculature of the patient, tab 214 is configured to allow the user to “slide” insertion tool 136 in the proximal direction along catheter 108 to remove insertion tool 136 from the introducer sheath and/or from catheter 108.

Housing 202 may not be slidable proximally over a manifold or other hub at a proximal end of catheter 108. Rather than requiring a clinician to maintain insertion tool 136 over catheter 108 during a medical procedure, in some examples, housing 202 is configured to be removed from catheter 108 in a direction transverse to longitudinal axis 216 of housing 202. For example, housing 202 can be configured to be longitudinally splitable to facilitate removal in the direction transverse to longitudinal axis 216 of housing 202. As an example, housing 202 can be preformed to longitudinally split without the aid of a sharp cutting tool. In addition, tab 214 can be configured to facilitate longitudinal splitting of housing 202. The longitudinal splitting can result in one longitudinal opening or can result in more than one longitudinal opening, which can cause housing 202 to split into multiple portions.

In some examples, housing 202 is configured to be preferentially longitudinally split along one or more predetermined paths. The preferential splitting may enable a clinician to better predict how housing 202 will operate during use, which may allow the clinician to better orient the housing during a medical procedure. For example, housing 202 includes peel line 218 configured to allow housing 202 to open to insertion tool lumen 204 along longitudinal axis 216. In the example shown, peel line 218 is opposite aperture 212, however, in other examples peel line 218 may be at any position on housing 202, e.g., at any circumferential position about housing 202. Peel line 218 may comprise a groove, a slit, a scoring mark, a seam, a perforation or perforated line or portion extending in the longitudinal direction along housing 202, or any suitable peel line 218 configured to allow housing 202 to be opened along its length in the longitudinal direction to expose insertion tool lumen 204 and release catheter 108.

For example, a clinician may insert catheter 108 into the introducer sheath and/or vasculature of the patient via insertion tool 136. The clinician may then move insertion tool 136 in the proximal direction along and relative to catheter 108, removing it from the introducer sheath, and then remove insertion tool 136 from catheter 108 via opening the insertion tool along peel line 218. Although shown as a substantially straight peel line 218 along the length of housing 202, peel line 202 may also extend about the circumference of housing 202 in any manner, e.g., peel line 218 may follow a helical path along housing 202.

FIGS. 6-8 are conceptual side views of the example insertion tool 136 of FIGS. 2-5 with catheter 108 in in differing configurations relative to housing 202 of insertion tool 136. FIG. 6 is a conceptual side view of insertion tool 136 with catheter 108 inserted in insertion tool 136 in a storage and/or pre-loaded configuration, FIG. 7 is a conceptual side view of insertion tool 136 with catheter 108 inserted in insertion tool 136 in a retracted configuration, and FIG. 8 is a conceptual side view of insertion tool 136 with catheter 108 inserted in insertion tool 136 in a bypass configuration.

As shown in FIG. 6, distal portion 302 of catheter 108 defines a bent 204, also referred to herein as a curve 304 in some examples. In the example shown, distal catheter portion 302 includes first distal portion 306 and second distal portion 308, with a curve 304 therebetween. First distal portion 306 is proximal to curve 304 and second distal portion 308 is distal to curve 304 and extends out of insertion tool lumen 204 via aperture 212.

Catheter 108, e.g., distal catheter portion 302, may be inserted into insertion tool 136 through proximal opening 206 and distally advanced through insertion tool lumen 204. If second distal portion 308 is curved toward sidewall 210 and aperture 212, then second distal portion 308 may exit through aperture 212 as catheter 108 is distally advanced through insertion tool lumen 204. Catheter 108 may continue to be distally advanced such that second distal portion 308 extends out of insertion tool lumen 204 via aperture 212 and curve 304 is unconstrained. For example, distal advancement of catheter 108 may cease before second distal portion 308 contacts and/or presses against a portion of sidewall 210 distal to aperture 212, e.g. at or about distal sidewall portion 220 illustrated in FIG. 6.

In some examples, insertion tool 136 is configured to enable distal catheter portion 302 to be rotated, e.g., about longitudinal axis 216 of insertion tool 136, within insertion tool lumen 204 and distally and/or proximally advanced within insertion tool lumen 204, e.g., to exit insertion tool lumen 204 via aperture 212 or to bypass aperture 212 and exit insertion tool lumen 204 via distal opening 208. For example, if second distal portion 308 is not curved toward sidewall 210 and aperture 212 after being inserted into insertion tool 136, then second distal portion 308 and/or insertion tool 136 may be rotated, such that second distal portion 308 is curved towards sidewall 210 and aperture 212 and distally advanced to extend second distal portion 308 out of insertion tool lumen 204 via aperture 212 as described above. If second distal portion 308 is not curved toward sidewall 210 and aperture 212 and second distal portion 308 is not rotated, then second distal portion 308 may not exit through aperture 212 as catheter 108 is distally advanced and may instead exit through distal opening 208.

Alternatively, a proximal end of catheter 108 may be inserted into insertion tool 136 through distal opening 208 and proximally advanced through insertion tool lumen (or insertion tool 136 may be distally advanced along catheter 108) until distal catheter portion 302 is within insertion tool lumen 204. If needed, a clinician can rotate catheter 108 and housing 202 relative to each other to align second distal portion 308 with aperture 212 as described above, and if needed, catheter 108 may be distally advanced once second distal portion 308 exits aperture 308 such that catheter 108 and insertion tool 136 are in the pre-loaded configuration with curve 304 in a relaxed, e.g., unconstrained, state.

In some examples, sidewall 210 and aperture 212 are configured to releasably hold distal portion 302 in a relaxed state, e.g., with curve 304 unconstrained, and to allow second distal portion 308 of catheter 108 extending out of insertion tool lumen 204 to be withdrawn proximally into insertion tool lumen 204. For example, aperture 212 may be sized such that second distal portion 308 is interference fit with aperture 212 and/or catheter 108 may be interference fit with insertion tool lumen 204. For example, an inner diameter of insertion tool lumen 204 may be substantially the same as (e.g., equal to or nearly equal to as permitted by manufacturing tolerances) an outer diameter of catheter 108, e.g., resulting in an interference fit that resists relative motion of catheter 108 and housing 202 but still enables catheter 108 to move within insertion tool lumen 204 with the application of a threshold amount of force (e.g., in the longitudinal direction) to catheter 108 relative to insertion tool 136 (e.g., to hold one of insertion tool 136 or catheter 108 steady and to move the other of insertion tool 136 or catheter 108).

In other examples, an inner diameter of insertion tool 204 may be larger than an outer diameter of catheter 108. In some examples, aperture 212 may be sized slightly larger than an outer diameter and/or perimeter of second distal portion 308 such that, in the pre-loaded configuration with second distal portion 308 extending out of insertion tool lumen 204 via aperture 212 with curve 304 unconstrained, distal or proximal movement of catheter 108 results in contact between second distal portion 308 and sidewall 212 (e.g., an edge of aperture 212 defined by sidewall 210) resisting further movement. For example, an amount of force to further bend curve 304, or bend catheter 108 at a position proximal to curve 304, would be required to distally advance catheter 108 beyond its position in the pre-loaded configuration. Also, a threshold amount of force to both overcome the friction of contact between second distal portion 308 and sidewall 210 and to straighten curve 304 would be required to proximally retract catheter 108 from its position in the pre-loaded configuration.

Insertion tool 136 is configured to constrain catheter 108 to reduce an amount of curvature of the curve upon distal portion 302 and/or second distal portion 308 of catheter 108 being withdrawn into insertion tool lumen 204 and to allow distal portion 302, when distal portion 302 and/or second distal portion 308 of catheter 108 is withdrawn into insertion tool lumen 204, to be rotated within insertion tool lumen 204 and then distally advanced within insertion tool lumen 204 to bypass aperture 212 and to exit insertion tool lumen 204 through distal opening 208. For example, a clinician may exert a threshold force on catheter 108 to proximally move distal portion 302 (e.g., to pull catheter 108 proximally) from the pre-loaded configuration by a distance such that at least a portion of distal portion 302 is within insertion tool lumen 204. Curve 304 may be configured to straighten (fully or partially) with the application of the threshold force, e.g., to allow second distal portion 308 to enter and be within insertion tool lumen 204.

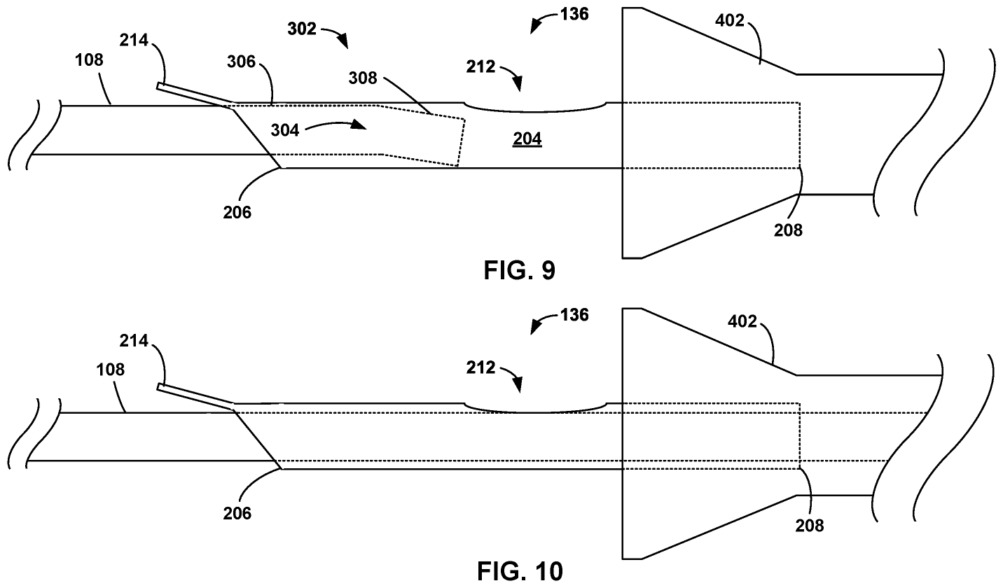

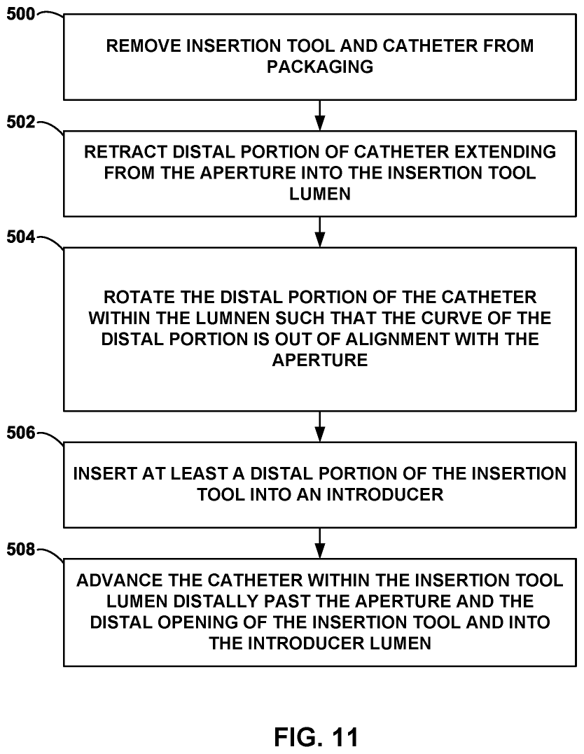

FIG. 11 is a flow diagram of an example method of using insertion tool 136. The techniques of FIG. 11 are primarily described with reference to insertion tool 136 and catheter 108 of the medical system 100 and introducer 402, however, in other examples, the technique may also be used with other insertion tools, catheters, introducers and/or introducer tools, in accordance with examples described herein. FIGS. 6-10 illustrate configurations of insertion tool 136 and catheter 108 at different steps and/or stages of the method of FIG. 11 and are described in conjunction with FIG. 11. FIG. 9 is a conceptual side view of insertion tool 136 with catheter 108 positioned in insertion tool lumen 204 and rotated to be in the bypass configuration of FIG. 8, and with insertion tool 136 inserted into a lumen of an introducer 402. FIG. 10 is a conceptual side view of insertion tool 136 with catheter 108 advance through insertion tool 136 and into introducer 402.

A user (e.g., a clinician) removes insertion tool 136 and catheter 108 in the pre-loaded configuration, e.g., as illustrated in FIG. 6, from packaging (500). The user then retracts distal portion 302 of catheter 108 extending from aperture 212 into insertion tool lumen 204 (502). For example, the user may grip tab 214 and pull on catheter 108 to move catheter 108 proximally relative to insertion tool 136 by a distance such that at least a portion of distal portion 302 is within insertion tool lumen 204. In addition to or instead of moving catheter 108 relative to insertion tool 136, the user may move insertion tool 136 relative to catheter 108. Insertion tool 136 and catheter 108 may then be in the retracted configuration as illustrated in FIG. 7.

The user then rotates catheter 108, e.g., relative to insertion tool 136, to rotate distal portion 302 within insertion tool lumen 204 such that curve 304 and/or second distal portion 308 are out of alignment with aperture 212 (504). For example, the user may rotate catheter 108 about longitudinal axis 216 and relative to insertion tool 136 by R as illustrated in FIG. 8, e.g., in either the clockwise or counterclockwise directions. The insertion tool 136 and catheter 108 may then be in the bypass configuration as illustrated in FIG. 9, in which distal catheter portion 302 will bypass aperture 302 as catheter 108 moves distally through insertion tool lumen 204 and out of distal opening 208 of insertion tool housing 202.

The user then inserts at least a distal portion of housing 202 of insertion tool 136 into a lumen of introducer 402 (506), e.g., as illustrated in FIG. 9. For example, the user may insert a distal portion of housing 202 defining distal opening 208 into a lumen of introducer sheath 402 through a valve of introducer sheath 402. Introducer sheath 402 can be a different type of medical device in other examples, such as another insertion tool, a different type of sheath, or the like.

The user may insert the distal portion of insertion tool 136 such that distal opening 208 is inserted beyond the valve of introducer sheath 402. Introducer tool housing 202 and introducer sheath 402 may be configured such that the valve contacts the outer surface of housing 202 to seal the lumen of introducer sheath 402 and help minimize or prevent fluid from leaving a lumen of introducer sheath 402 around insertion tool housing 202. In the example shown in FIGS. 9 and 10, the user inserts insertion tool 136 such that aperture 212 is outside of the lumen of introducer 402. In other examples, aperture 212 is positioned, e.g., along the length of housing 202, to be within the lumen of introducer 402 when the distal end and/or portion of housing 402 is inserted and/or received within the lumen of introducer 402. For example, the user may insert insertion tool 136 such that aperture 212 is with of the lumen of introducer 402, e.g., aperture 212 is inserted beyond a seal of introducer 402. In some examples, the user may insert insertion tool 136 by pressing insertion tool into the introducer lumen of introducer 402 for an interference fit. In some examples, the user may insert insertion tool 136 into introducer 402 before rotating catheter 108 (504), or even retracting catheter 108 (502), e.g., the steps shown in FIG. 11 may occur in any suitable order.

The user then advances catheter 108 (and distal portion 302) within insertion tool lumen 204 advance distal portion 302 distally past aperture 212 and distal opening 208 and into introducer 402 (508). In some examples, the user advances distal portion into the introducer lumen of introducer 402 distally past a seal of introducer 402.

In some examples, the user then retracts housing 202 from the introducer lumen of introducer 402 along catheter 108 while keeping distal portion 302 of catheter 108 advanced distally past the valve and within the introducer lumen. For example, the user may grip tab 214 and catheter 108 and move insertion tool 136 proximally along, and relative to, catheter 108. The user may then remove insertion tool 136, e.g., housing 202, from catheter 108. For example, the user may remove catheter 108 from insertion tool lumen of housing 202 in a direction transverse to longitudinal axis 216 of housing 202 via peel line 218, which may be at least one of a groove, a slit, a scoring mark, a seam, a perforation, or the like formed in housing 202.

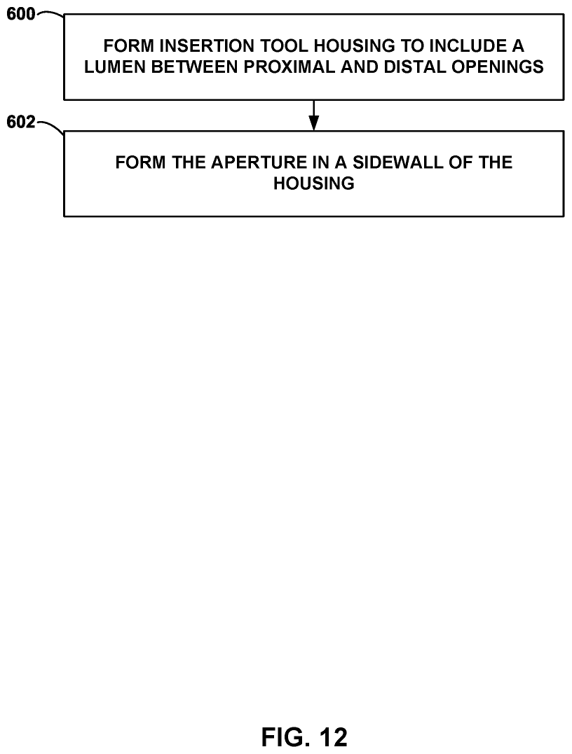

Insertion tool 216 can be formed form any suitable technique. FIG. 12 is a flow diagram of an example method of making insertion tool 136. The techniques of FIG. 12 are primarily described with reference to insertion tool 136 and catheter 108 of the medical system 100, however, in other examples, the technique may also be used with other insertion tools and catheters, in accordance with examples described herein.

A manufacturer forms housing 202 comprising proximal opening 206, distal opening 208, and insertion lumen 204 therebetween (600). The manufacturer forms housing 202 such that proximal opening 206 and distal opening 208 are configured to receive catheter 108 into insertion lumen 204 and the distal end of the housing including distal opening 208 is configured to be received within an introducer lumen of at least one of an introducer sheath or an introducer valve, e.g., introducer 402. In some examples, the manufacturer may form housing out of at least one of a polymer material, e.g., nylon, polycarbonate, polyester, polypropylene, a vinyl, a thermoset plastic, or the like, or an alcohol, or a metal.

The manufacturer forms aperture 212 in sidewall 210 of insertion tool 136 positioned between proximal opening 206 and distal opening 208 (602). In some examples, the manufacturer forms aperture 212 to be configured to receive a at least a portion of catheter 108 into or out of insertion tool lumen 204. In some example, the manufacturer may alternatively form sidewall 210 to define aperture 212, e.g., via the use of a spacer, tool, plug, or the like used while forming housing 202 in order to create the opening and/or void in sidewall 210 defining aperture 212. In other examples, the manufacturer may form aperture 212 by cutting, punching, and/or skiving or the like, sidewall 210.

In some examples, the manufacturer forms tab 214 such that tab 214 extends away from longitudinal axis 216 of housing 202 and such that tab 214 is configured to be gripped by a hand of a user to control a motion of housing when being gripped. In some examples, the manufacturer forms peel line 218, e.g., at least one of a groove, a slit, a scoring mark, a seam, or a perforation configured to enable housing 202 to be opened to insertion tool lumen 204 along its length to release catheter 108.

The following examples are within the scope of the present disclosure. The examples described herein may be combined in any permutation or combination.

Example 1: A kit including: a catheter; and an insertion tool including an insertion tool housing defining: an insertion tool lumen configured to receive at least a portion of the catheter, a proximal opening to the insertion tool lumen, and a distal opening to the insertion tool lumen, the insertion tool housing comprising a sidewall extending between the proximal and distal openings, wherein the sidewall defines an aperture, and when the at least the portion of the catheter is positioned in the insertion tool lumen, a distal portion of the catheter is configured to extend out of the insertion tool lumen via the aperture.

Example 2: The kit of example 1, wherein the insertion tool housing comprises at least one of a polymer or a metal.

Example 3: The kit of example 1 or example 2, wherein the insertion tool housing comprises at least one of nylon, polycarbonate, polyester, polypropylene, a alcohol, a vinyl, or a thermoset plastic.

Example 4: The kit of any one of examples 1-3, wherein the insertion tool housing further comprises a member extending from the insertion tool housing and configured to be gripped by a hand of a user to control a motion of the insertion tool housing.

Example 5: The kit of example 4, wherein the member is positioned at a proximal end of the insertion tool housing.

Example 6: The kit of example 5, wherein the member extends away from a longitudinal axis of the insertion tool housing.

Example 7: The kit of any one of examples 1-6, wherein when the at least a portion of the catheter is received in the insertion tool lumen, the insertion tool housing is configured to be removed from around the catheter in a direction transverse to a longitudinal axis of the insertion tool housing.

Example 8: The kit of example 7, wherein the insertion tool housing is configured to be opened along its length to expose the insertion tool lumen and enable removal of the catheter in the direction transverse to the longitudinal axis.

Example 9: The kit of example 8, wherein the insertion tool housing defines at least one of a groove, a slit, a scoring mark, a seam, or a perforation configured to enable the insertion tool housing to be opened along its length.

Example 10: The kit of any one of examples 1-9, further comprising an introducer defining an introducer lumen, wherein at least a distal portion of the insertion tool housing is configured to be received within the introducer lumen, and wherein when the insertion tool housing is fully inserted into the introducer lumen, the aperture is positioned within the introducer lumen.

Example 11: The kit of example 10, wherein the introducer lumen is sized and the insertion tool housing is configured such that the insertion tool housing is not compressed radially inward when received within the introducer lumen.

Example 12: The kit of example 10 or example 11, wherein the introducer comprises a valve within the introducer lumen, wherein the insertion tool housing is configured to open the valve when the insertion tool housing is inserted through the valve.

Example 13: The kit of any one of examples 1-12, wherein the aperture has an elliptical cross-sectional shape.

Example 14: The kit of example 13, wherein a length of the aperture along a longitudinal axis of the insertion tool housing is greater than a width of the aperture in a width direction transverse to the longitudinal axis.

Example 15: The kit of any one of examples 1-14, wherein a stiffness of the insertion tool housing is greater than a stiffness of the catheter.

Example 16: The kit of any one of examples 1-15, wherein an inner diameter of the insertion tool lumen is substantially the same as an outer diameter of the catheter.

Example 17: The kit of any one of examples 1-16, wherein the insertion tool housing is at least partially transparent or translucent.

Example 18: The kit of any one of examples 1-17, wherein a longitudinal length of the insertion tool housing is at least 40 millimeters.

Example 19: The kit of any one of examples 1-18, wherein an outer diameter of a distal portion of the insertion tool housing is tapered such that an outer diameter of the distal end of the distal portion is less than an outer diameter of the insertion tool housing at a longitudinal position proximal to the distal end of the insertion tool housing.

Example 20: A medical device package including: the catheter of any of claims 1-19; the insertion tool of any of claims 1-19; and a package housing, wherein the catheter and the catheter insertion tool are disposed within the package housing, and wherein, within the package housing, the at least the portion of the catheter is received within the insertion tool lumen and the distal portion of the catheter is extending out of the insertion tool lumen via the aperture.

Example 21: The medical device package of example 20, wherein the catheter and insertion tool are sterilized, and wherein the package housing is a sterile package housing.

Example 22 The medical device package of example 20 or example 21, wherein at least a part of the distal portion of the catheter defines a preformed curve, wherein the at least the part of the distal portion curves from the insertion tool lumen in a direction towards the aperture:

Example 23: The medical device package of example 22, wherein the preformed curve is unconstrained when, within the medical device package, the at least the portion of the catheter is received within the insertion tool lumen and the distal portion of the catheter is extending out of the insertion tool lumen via the aperture.

Example 24: The medical device package of example 22 or example 23, wherein the insertion tool is configured to enable the distal portion of the catheter extending out of the insertion tool lumen to be withdrawn proximally into the insertion tool lumen of the insertion tool.

Example 25: The medical device package of example 24, wherein the insertion tool is configured to constrain the catheter to reduce an amount of curvature of the curve when the distal portion of the catheter is withdrawn into the insertion tool lumen of the catheter.

Example 26: The medical device package of example 24 or example 25, wherein the insertion tool is configured to allow the distal portion of the catheter, when the distal portion of the catheter is withdrawn within the insertion tool lumen of the insertion tool, to be rotated within the insertion tool lumen and then distally advanced within the insertion tool lumen to bypass the aperture and to exit the insertion tool lumen through the distal opening.

Example 27: A method of making a catheter insertion tool, the method including: forming an insertion tool housing comprising a proximal opening, a distal opening, and an insertion tool lumen therebetween, the insertion tool lumen being configured to receive at least a portion of a catheter, wherein a distal end of the insertion tool housing is configured to be received within an introducer lumen of an introducer sheath; and forming an aperture in a sidewall of the insertion tool, the sidewall extending between the proximal and distal openings, wherein the aperture is configured to receive a portion of the catheter to enable the catheter to extend from the insertion tool lumen to an environment external to the insertion tool lumen.

Example 28: The method of example 27, wherein the insertion tool housing comprises at least one of a nylon, a polycarbonate, a polyester, a polypropylene, a polyvinyl alcohol, a vinyl, or a thermoset plastic.

Example 29: The method of example 27 or example 28, wherein forming the aperture comprises skiving the aperture in the sidewall of the insertion tool housing.

Example 30: The method of any one of examples 27-29, further includes forming a member that extends away from a longitudinal axis of the insertion tool housing, the member configured to be gripped by a hand of a user to control a motion of the housing insertion tool.

Example 31: The method of any one of examples 27-30, further includes forming at least one of a groove, a slit, a scoring mark, a seam, or a perforation in the insertion tool housing to enable the insertion tool housing to be opened along its length to release the catheter.

Example 32: A method includes retracting a distal portion of a catheter extending through an aperture in a sidewall of an insertion tool housing of an insertion tool into an insertion tool lumen defined by the insertion tool housing, wherein the distal portion of the catheter comprises a curve; rotating the catheter to rotate the distal portion of the catheter within the insertion tool lumen such that the curve is out of alignment with the aperture; inserting a distal portion of the insertion tool housing into an introducer lumen of an introducer sheath; and advancing the catheter within the insertion tool lumen, distally past the aperture, and into the introducer lumen.

Example 33: The method of example 32, further includes retracting the insertion tool housing from the introducer lumen along the catheter while keeping the distal portion of the catheter in the introducer lumen.

Example 34: The method of example 33, further comprising removing the insertion tool housing from the catheter.

Example 35: The method of example 34, wherein removing the insertion tool housing from the catheter comprises: removing the catheter from the lumen of the insertion tool housing in a direction transverse to a longitudinal axis of the insertion tool housing.

Example 36: The method of example 35, wherein removing the catheter from the lumen of the insertion tool housing in the direction comprises splitting the insertion tool housing along the longitudinal axis.

Example 37: A catheter insertion tool including: a housing defining a lumen includes a proximal opening to the lumen, the proximal opening being configured to receive a portion of a catheter into the lumen, a distal opening to the lumen configured to receive the portion of the catheter, an aperture in the sidewall of the lumen, the aperture being positioned between the proximal and distal openings, wherein when the portion of the catheter is positioned in the insertion tool lumen, a distal portion of the catheter is configured to extend out of the insertion tool lumen via the aperture, wherein the housing is configured to be removed from the catheter received within the insertion tool lumen in a direction transverse to a longitudinal axis of the catheter, wherein a distal portion of the housing including the distal opening is configured to be received within an introducer lumen of at least one of an introducer sheath.

Example 38: The catheter insertion tool of example 37, wherein the housing comprises at least one of a polymer or a metal.

Example 39: The catheter insertion tool of example 37 or example 38, wherein the housing comprises at least one of nylon, polycarbonate, polyester, polypropylene, a alcohol, a vinyl, or a thermoset plastic.

Example 40: The catheter insertion tool of any one of examples 37-39, wherein the housing further comprises a member extending from the housing and configured to be gripped by a hand of a user to control a motion of the housing.

Example 41: The catheter insertion tool of example 40, wherein the member is positioned at a proximal end of the housing.

Example 42: The catheter insertion tool of example 41, wherein the member extends away from a longitudinal axis of the housing.

Example 43: The catheter insertion tool of example 42, wherein the housing is configured to be opened along its length in the longitudinal direction to expose the lumen and release the catheter.

Example 44: The catheter insertion tool of example 43, wherein the housing comprises at least one of a groove, a slit, a scoring mark, a seam, or a perforation configured to enable the housing to be opened along its length.

Example 45: The catheter insertion tool of any one of examples 37-44, wherein the aperture has an elliptical cross-sectional shape.

Example 46: The catheter insertion tool of example 47, wherein a length of the aperture along a longitudinal axis of the housing is greater than a width of the aperture in a width direction perpendicular to the longitudinal axis.

Example 47: The catheter insertion tool of any one of examples 37-46, wherein a stiffness of the housing is greater than a stiffness of the catheter.

Example 48: The catheter insertion tool of any one of examples 37-47, wherein the housing is configured to open a valve within an introducer lumen of an introducer sheath when the housing is inserted through the valve.

Example 49: The catheter insertion tool of any one of examples 37-48, wherein the housing is at least one of transparent or translucent.

Example 50: The catheter insertion tool of any one of examples 37-49, wherein a longitudinal length of the housing is at least 40 millimeters.

Example 51: The catheter insertion tool of any one of examples 37-50, wherein an outer diameter of a distal portion of the housing is tapered in a distal direction.

Various aspects of the disclosure have been described. These and other aspects are within the scope of the following claims.

1. A kit comprising:
a catheter; and
an insertion tool including an insertion tool housing defining:
an insertion tool lumen configured to receive at least a portion of the catheter,
a proximal opening to the insertion tool lumen, and
a distal opening to the insertion tool lumen,
the insertion tool housing comprising a sidewall extending between the proximal and distal openings, wherein the sidewall defines an aperture, and when the at least the portion of the catheter is positioned in the insertion tool lumen, a distal portion of the catheter is configured to extend out of the insertion tool lumen via the aperture.

2. The kit of claim 1, wherein the insertion tool housing further comprises a member extending from the insertion tool housing and configured to be gripped by a hand of a user to control a motion of the insertion tool housing.

3. The kit of claim 2, wherein the member is positioned at a proximal end of the insertion tool housing, and wherein the member extends away from a longitudinal axis of the insertion tool housing.

4. The kit of claim 1, wherein when the at least a portion of the catheter is received in the insertion tool lumen, the insertion tool housing is configured to be removed from around the catheter in a direction transverse to a longitudinal axis of the insertion tool housing.

5. The kit of claim 4, wherein the insertion tool housing is configured to be opened along its length to expose the insertion tool lumen and enable removal of the catheter in the direction transverse to the longitudinal axis.

6. The kit of claim 1, further comprising an introducer defining an introducer lumen, wherein at least a distal portion of the insertion tool housing is configured to be received within the introducer lumen, and wherein when the insertion tool housing is fully inserted into the introducer lumen, the aperture is positioned within the introducer lumen.

7. The kit of claim 1, wherein a length of the aperture along a longitudinal axis of the insertion tool housing is greater than a width of the aperture in a width direction transverse to a longitudinal axis of the insertion tool housing.

8. The kit of claim 1, wherein a stiffness of the insertion tool housing is greater than a stiffness of the catheter.

9. The kit of claim 1, wherein the insertion tool housing is at least partially transparent or translucent.

10-14. (canceled)

15. A catheter insertion tool comprising:
a housing defining a lumen, the housing comprising a sidewall and defining:
a proximal opening to the lumen, the proximal opening being configured to receive a portion of a catheter into the lumen,
a distal opening to the lumen configured to receive the portion of the catheter,
an aperture in the sidewall of the lumen, the aperture being positioned between the proximal and distal openings,
wherein when the portion of the catheter is positioned in the insertion tool lumen, a distal portion of the catheter is configured to extend out of the insertion tool lumen via the aperture,
wherein the housing is configured to be removed from the catheter received within the insertion tool lumen in a direction transverse to a longitudinal axis of the catheter,
wherein a distal portion of the housing including the distal opening is configured to be received within an introducer lumen of at least one of an introducer sheath.

16. The catheter insertion tool of claim 15, wherein the housing comprises at least one of a polymer or a metal.

17. The catheter insertion tool of claim 15, wherein the housing comprises at least one of nylon, polycarbonate, polyester, polypropylene, a alcohol, a vinyl, or a thermoset plastic.

18. The catheter insertion tool of claim 15, wherein the housing further comprises a member extending from the housing and configured to be gripped by a hand of a user to control a motion of the housing.

19. The catheter insertion tool of claim 18, wherein the member is positioned at a proximal end of the housing.

20. The catheter insertion tool of claim 19, wherein the member extends away from a longitudinal axis of the housing.

21. The catheter insertion tool of claim 20, wherein the housing is configured to be opened along its length in the longitudinal direction to expose the lumen and release the catheter.

22. The catheter insertion tool of claim 21, wherein the housing comprises at least one of a groove, a slit, a scoring mark, a seam, or a perforation configured to enable the housing to be opened along its length.

23. The catheter insertion tool of claim 15, wherein the aperture has an elliptical cross-sectional shape.

24. The catheter insertion tool of claim 15, wherein a length of the aperture along a longitudinal axis of the housing is greater than a width of the aperture in a width direction perpendicular to the longitudinal axis.

25. A method comprising:
retracting a distal portion of a catheter extending through an aperture in a sidewall of an insertion tool housing of an insertion tool into an insertion tool lumen defined by the insertion tool housing, wherein the distal portion of the catheter comprises a curve;
rotating the catheter to rotate the distal portion of the catheter within the insertion tool lumen such that the curve is out of alignment with the aperture;
inserting a distal portion of the insertion tool housing into an introducer lumen of an introducer sheath; and
advancing the catheter within the insertion tool lumen, distally past the aperture, and into the introducer lumen.
